(19) United States
(12) Patent Application Publication
(10) Pub. No.: US 2021/0332014 A1
(43) Pub. Date: Oct. 28, 2021

(54) CABOZANTINIB MALATE CRYSTAL FORM, PREPARATION METHOD AND USE THEREOF

(71) Applicant: Crystal Pharmaceutical (Suzhou) Co., Ltd., Suzhou (CN)

(72) Inventors: Minhua Chen, Suzhou (CN); Yanfeng Zhang, Suzhou (CN); Xiaoting Zhai, Suzhou (CN); Jing Zhang, Suzhou (CN); Qun Zhang, Suzhou (CN)

(21) Appl. No.: 17/278,138

(22) PCT Filed: Sep. 20, 2019
(86) PCT No.: PCT/CN2019/106847

§ 371 Date: Mar. 19, 2021

(30) Foreign Application Priority Data
Sep. 20, 2018 (CN) 201811096899.6

(51) Int. Cl. C07D 215/233

(52) U.S. Cl. CPC C07D 215/233; C07B 2200/13

(57) ABSTRACT

The present invention relates to novel cabozantinib malate crystalline forms, preparation methods for the cabozantinib malate, a pharmaceutical composition comprising the novel cabozantinib malate crystalline forms, and use of the novel cabozantinib malate crystalline forms in the preparation of MET, VEGFR1/2/3, ROS1, RET, AXL, NTRK, and KIT inhibitors and pharmaceutical preparations for treating cancers such as thyroid cancer, lung cancer, kidney cancer and liver cancer. The cabozantinib malate crystalline forms provided by the present invention has one or more improved properties compared with the prior art, and the preparation method for the cabozantinib malate provided by the present disclosure has a lower cost and better quality of the obtained product compared with the prior art, having important value for future optimization and development of this drug.

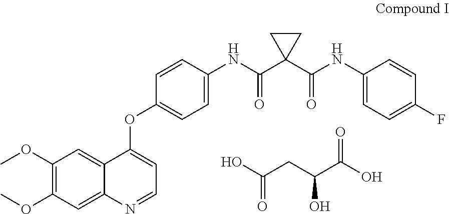

### TECHNICAL FIELD

The present disclosure relates to the field of pharmaceutical chemistry, particularly relates to crystalline forms of cabozantinib malate, processes for preparing and use thereof.

### BACKGROUND

Cabozantinib is an anticancer drug developed by Exelixis, and it was approved by FDA in November 2012 and April 2016 for the treatment of metastatic medullary thyroid cancer and renal cell carcinoma, respectively. In addition, the indications for the treatment of liver cancer was also approved by FDA in January 2019. Cabozantinib is marketed as (S)-malate.

The chemical name of cabozantinib (S)-malate is N-(4-(6,7-dimethoxyquinolin-4-yloxy) phenyl)-N′-(4-fluorophenyl) cyclopropane-1,1-dicarboxamide (S)-malate (hereinafter referred to as “Compound I” or cabozantinib (S)-malate). Its structural formula is as follows:

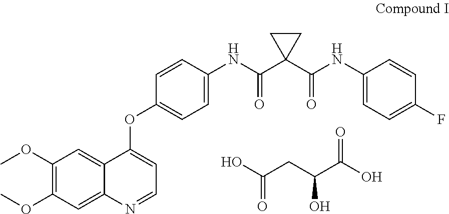

A crystalline form is a solid material whose constituents are arranged in a highly ordered microscopic structure, forming a crystal lattice that extends in all directions. Polymorphism is the ability of a compound to exist in two or more than two crystalline forms. Different crystalline forms have different physicochemical properties and can affect drug's in vivo dissolution and absorption, which will further affect drug's clinical efficacy and safety to some extent. In particular, for poorly soluble drugs, the above effects of the crystalline form will be greater. Therefore, drug polymorphism is an important part of drug research and an important part of drug quality control.

At present, although there are reports on the crystalline forms of Compound I, the properties of the crystalline forms that have been reported are not good, and there are still some problems. For example, CN102388024A disclosed the crystalline form N-1, crystalline form N-2, and amorphous of Compound I. CN102388024A shows that crystalline form N-2 has better stability than amorphous and crystalline form N-1. However, the solubility of crystalline form N-2 is low, and the flowability, compressibility, tensile strength, and adhesion are poor. WO2015177758A1 disclosed crystalline form M1, crystalline form M2, crystalline form M3 and crystalline form M4 of Compound I, wherein crystalline form M4 is better. However, crystalline form M4 has low solubility, poor fluidity, poor compressibility, poor tensile strength, and poor adhesion. Therefore, a large number of experimental studies are still needed to provide more crystal forms with better properties to support the development of Compound I drugs.

In order to overcome the disadvantages of prior art, the inventors of the present disclosure surprisingly discovered crystalline form CSI and crystalline form CSIII of Compound I, which has advantages in physiochemical properties, formulation processability and bioavailability, for example, crystalline form CSI and crystalline form CSIII have advantages in at least one aspect of melting point, solubility, hygroscopicity, purification ability, stability, adhesiveness, compressibility, flowability, in vitro and in vivo dissolution, and bioavailability, etc. Particularly, crystalline form CSI and crystalline form CSIII have high solubility and good fluidity, tensile strength and adhesion, which provides new and better choices for the development of drugs containing Compound I, and is of great significance.

In addition, when the inventors studied the crystal forms of the prior art, it was found that the preparation method of the crystalline form M2 disclosed by WO2015177758 A1 (hereinafter referred to as “Form M2”) has poor repeatability and it is difficult to control the process.

Therefore, developing a robust and controllable preparation method of crystalline form M2 is also of great value for the development of Compound I drugs.

### SUMMARY

The main objective of the present disclosure is to provide novel crystalline forms of Compound I and processes for preparation and use thereof.

According to the objective of the present disclosure, crystalline form CSI of Compound I is provided (hereinafter referred to as Form CSI).

According to one aspect of the present disclosure, the X-ray powder diffraction pattern of Form CSI shows characteristic peaks at 2 theta values of 8.5°±0.2°, 12.7°±0.2°, 13.9°±0.2° using CuKα radiation.

Furthermore, the X-ray powder diffraction pattern of Form CSI shows one or two or three characteristic peaks at 2 theta values of 12.1°±0.2°, 17.9°±0.2°, 19.9°±0.2°. Preferably, the X-ray powder diffraction pattern of Form CSI shows three characteristic peaks at 2 theta values of 12.1°±0.2°, 17.9°±0.2°, 19.9°±0.2°.

Furthermore, the X-ray powder diffraction pattern of Form CSI shows one or two or three characteristic peaks at 2 theta values of 14.9°±0.2°, 16.7°±0.2°, 25.5°±0.2°. Preferably, the X-ray powder diffraction pattern of Form CSI shows three characteristic peaks at 2 theta values of 14.9°±0.2°, 16.7°±0.2°, 25.5°±0.2°.

According to another aspect of the present disclosure, the X-ray powder diffraction pattern of Form CSI shows three or four or five or six or seven or eight or nine characteristic peaks at 2 theta values of 8.5°±0.2°, 12.7°±0.2°, 13.9°±0.2°, 12.1°±0.2°, 17.9°±0.2°, 19.9°±0.2°, 14.9°±0.2°, 16.7°±0.2°, 25.5°±0.2° using CuKα radiation.

Furthermore, Form CSI is an acetic acid solvate.

Without any limitation being implied, the X-ray powder diffraction pattern of Form CSI is substantially as depicted in FIG. 1.

According to the objective of the present disclosure, a process for preparing Form CSI is also provided. The process includes the following methods:

Method 1: dissolving Compound I in acetic acid or a solvent mixture of acetic acid and an aromatic hydrocarbon, then fast evaporating at 50-80° C.;

Method 2: dissolving the Compound I solid in acetic acid, a mixture of acetic acid and an aromatic, a mixture of acetic acid and an alkane, or a mixture of acetic acid and water, then adding an aromatic hydrocarbon, an alkane, an ester or a ketone into the solution with stirring. The obtained solid is crystalline form CSI.

Furthermore, said aromatic hydrocarbons in method 1 is toluene; the volume ratio of said acetic acid and toluene is 2:1-1:3, preferably 1:1.

Furthermore, the volume ratios of said acetic acid and aromatic hydrocarbon, said acetic acid and alkane, or said acetic acid and water in method 2 are 2:1-1:3, preferably 1:1.

Furthermore, in method 2, said aromatic hydrocarbon is toluene, said alkane is n-heptane, said ester is isopropyl acetate, and said ketone is methyl isobutyl ketone.

Furthermore, said stirring in method 2 is performed at 0-5° C.

According to the objective of the present disclosure, crystalline form CSIII of Compound I is provided (hereinafter referred to as Form CSIII).

According to one aspect of the present disclosure, the X-ray powder diffraction pattern of Form CSIII shows characteristic peaks at 2 theta values of 8.5°±0.2°, 21.3°±0.2°, 23.0°±0.2° using CuKα radiation.

Furthermore, the X-ray powder diffraction pattern of Form CSIII shows one or two or three characteristic peaks at 2 theta values of 14.4°±0.2°, 17.8°±0.2°, 12.6°±0.2°. Preferably, the X-ray powder diffraction pattern of Form CSIII shows three characteristic peaks at 2 theta values of 14.4°±0.2°, 17.8°±0.2°, 12.6°±0.2°. Furthermore, the X-ray powder diffraction pattern of Form CSIII shows one or two or three characteristic peaks at 2 theta values of 20.5°±0.2°, 24.0°±0.2°, 16.4°±0.2°. Preferably, the X-ray powder diffraction pattern of Form CSIII shows three characteristic peaks at 2 theta values of 20.5°±0.2°, 24.0°±0.2°, 16.4°±0.2°.

According to another aspect of the present disclosure, the X-ray powder diffraction pattern of Form CSIII shows three or four or five or six or seven or eight or nine characteristic peaks at 2 theta values of 8.5°±0.2°, 21.3°±0.2°, 23.0°±0.2°, 14.4°±0.2°, 17.8°±0.2°, 12.6°±0.2°, 20.5°±0.2°, 24.0°±0.2°, 16.4°±0.2° using CuKα radiation.

Without any limitation being implied, the X-ray powder diffraction pattern of Form CSIII is substantially as depicted in FIG. 6.

According to the objective of the present disclosure, a process for preparing Form CSIII is also provided. The process includes the following methods:

Method 1: dissolving Compound I in an acid, a mixture of an acid and an aromatic hydrocarbon, a mixture of an acid and an alkane, or a mixture of an acid and water, adding aromatic hydrocarbon, alkane, ester or ketone into the solution with stirring to precipitate a solid. Filtrating to get the solid. The obtained solid was slurred in a solvent mixture of an aromatic hydrocarbon and water, separating the solid again to get Form CSIII;

Method 2:

step 1: dissolving Compound I in an acid, stirring and heating until the solid was completely dissolved, then naturally cooling to room temperature and filtering.

step 2: adding an aromatic hydrocarbon dropwise to the clear solution, transferring the mixture to an environment at 0-10° C. with continuous stirring, then filtering the mixture to separate the solid, and drying;

Step 3: heating the solid to 50-100° C. under nitrogen purging, and then cooling to 30° C., the obtained solid is Form CSIII.

Furthermore, the volume ratio of said acid and aromatic hydrocarbon, acid and alkane, or acid and water in Method 1 are 2:1-1:3, preferably, the volume ratio is 1:1.

Furthermore, in method 1, said acid is acetic acid, said aromatic hydrocarbon is toluene, said alkane is n-heptane, said ester is isopropyl acetate, and said ketone is methyl isobutyl ketone.

Furthermore, in method 2, said acid is acetic acid, said aromatic hydrocarbon is toluene.

Furthermore, in method 2, said stirring of step 1 is at 80° C.; said stirring of step 2 is at 5° C., the time of said stirring of step 2 is 10-20 hours, and said heating of step 3 is up to 100° C.

Further, in method 2, the time of said stirring of step 2 is 15 hours.

Form CSI of the present disclosure has the following advantages:

(1) Compared with prior art, Form CSI has higher solubility. In a specific embodiment, the solubility of Form CSI in water is twice of that of Form N-2 and over five times more than that of Form M4.

Cabozantinib is a poorly water-soluble drug and belongs to BCS class II. Higher solubility is beneficial to improve drug's in vivo absorption and bioavailability, thus improving drug efficacy. In addition, drug dose reduction without affecting efficacy is possible due to higher solubility, thereby reducing the drug's side effects and improving drug safety.

(2) Form CSI of the present disclosure has good purification effect. The purity is significantly increased after the raw material is converted to Form CSI of the present disclosure. In a specific embodiment, after Form CSI is prepared from the raw material using the crystallization process, the purity is significantly increased and the content of each impurity is reduced.

Chemical purity is of great significance for ensuring drug efficacy, safety and preventing the occurrence of adverse effects. If the drug contains impurities higher than limit, its physicochemical properties and drug appearance may change, and the stability will be affected. The increase in impurities will lead to significantly lowered active ingredient content or reduced drug activity, and will also lead to significantly increased toxicity and side effects of the drug products. Therefore, different drug regulations have strict requirements on impurity content. Crystalline forms with good purification effect are excellent in removing impurities in the crystallization process, thus drug substances with high purity can be obtained through crystallization, which effectively overcome the disadvantages of poor stability, poor efficacy and high toxicity caused by the low purity drug substances.

Form CSIII of the present disclosure has the following advantages:

Compared with prior art, Form CSIII has higher solubility. Especially in FeSSIF and water, the solubility of Form CSIII is twice of that of Form N-2 and Form M4.

Cabozantinib is a poorly water-soluble drug and belongs to BCS class II. Higher solubility is beneficial to improve drug's in vivo absorption and bioavailability, thus improving drug efficacy.

In addition, drug dose reduction without affecting efficacy is possible due to higher solubility, thereby reducing the drug's side effects and improving drug safety.

Furthermore, Form CSIII of the present disclosure also has the following advantages:

(1) Compared with prior art, Form CSIII of the present disclosure has better flowability. Flowability evaluation results indicate that the flowability of Form CSIII is remarkably better than that of prior art forms. Better flowability can prevent clogging of production equipment and increase manufacturing efficiency. Better flowability of Form CSIII ensures the blend uniformity and content uniformity of the drug product, and reduces the weight variation of the drug product and improves product quality

(2) Compared with prior art, Form CSIII of the present disclosure has better compressibility. Failure in hardness/friability test and tablet crack issue can be avoided due to better compressibility, making the preparation process more reliable, improving product appearance and product quality. Better compressibility can increase the compression rate, thus further increases the efficiency of process and reduces the cost of compressibility improving excipients.

(3) Compared with prior art, Form CSIII of the present disclosure shows superior adhesiveness. Adhesiveness evaluation results indicate that adhesion quantity of Form CSIII is remarkably lower than that of prior art forms. Due to superior adhesiveness of Form CSIII, adhesion to roller and tooling during dry-granulation and compression process can be reduced, which is also beneficial to improve product appearance and weight variation. In addition, Superior adhesiveness of Form CSIII can reduce the agglomeration of drug substance, which is beneficial to the dispersion of drug substance and reduce the adhesion between drug substance and other instruments, and improve the blend uniformity and content uniformity of drug product.

According to the objective of the present disclosure, a pharmaceutical composition is provided. Said pharmaceutical composition comprises a therapeutically effective amount of Form CSI or Form CSIII or combinations thereof and pharmaceutically acceptable carrier, dilution agents or excipients.

Furthermore, Form CSI or Form CSIII or combinations thereof can be used for preparing drugs inhibiting MET, VEGFR1/2/3, ROS1, RET, AXL, NTRK and KIT.

Furthermore, Form CSI or Form CSIII or combinations thereof can be used for preparing drugs treating thyroid cancer, lung cancer, gastric cancer, and liver cancer.

According to the objective of the present disclosure, a process for preparing Form M2 of Compound I is provided. The process comprises: dissolving the solid of Compound I or a solid mixture of cabozantinib and (S)-malic acid in solvent and then adding an anti-solvent to precipitate a solid, then drying the solid under a condition of more than 30% relative humidity (RH) to obtain Form M2. Said solvent is an organic acid or a solvent mixture of an organic acid and an aromatic hydrocarbon; said anti-solvent is an aromatic hydrocarbon, an ester, an alcohol, a ketone or a solvent mixture of an aromatic hydrocarbon and an ester or an aromatic hydrocarbon and a ketone; the X-ray powder diffraction pattern of said Form M2 shows characteristic peaks at 2 theta values of 8.6°±0.2°, 12.6°±0.2°, 20.2°±0.2°, 23.4°±0.2°, 26.1°±0.2.

Furthermore, said organic acid is acetic acid, said aromatic hydrocarbon is toluene, said ester is ethyl acetate or isopropyl acetate, said ketone is methyl isobutyl ketone, and said alcohol isopropanol or n-propanol.

Furthermore, the temperature of the solvent system when adding the anti-solvent is below 15° C.; preferably −5° C. to 10° C.

Furthermore, seed crystals of Form M2 can be added before adding the anti-solvent; the amount of seed crystals are 1 wt % to 10 wt %.

Furthermore, the volume ratio of the solvent and the anti-solvent is 1:1 to 1:10; preferably, the volume ratio is 2: 5.

Compared with the prior art, the process for preparing Form M2 provided by the present disclosure has advantages as it is controllable and can be scaled up easily. It can be seen from the comparative example that crystal form N-1 of CN102388024A instead of Form M2 is obtained by repeating the preparation method of the prior art. In addition, Form M2 obtained by the preparation method provided by the present disclosure has the advantages of high yield, low solvent residue, and uniform particle size distribution. Such a preparation method not only saves costs, but also provides high-quality drug substances, which provides new and better choices for preparation of drug product containing cabozantinib and has significant values for future drug development.

In the present disclosure, said “stirring” is accomplished by using a conventional method in the field such as magnetic stirring or mechanical stirring and the stirring speed is 50 to 1800 r/min, preferably the magnetic stirring speed is 300 to 900 r/min and mechanical stirring speed is 100 to 300 r/min.

Said “separation” is accomplished by using a conventional method in the field such as centrifugation or filtration. The operation of “centrifugation” is as follows: the sample to be separated is placed into the centrifuge tube, and then centrifuged at a rate of 10000 r/min until the solid all sink to the bottom of the tube.

Said “drying” is accomplished at room temperature or a higher temperature. The drying temperature is from room temperature to about 60° C., or to 40° C., or to 50° C. The drying time can be 2 to 48 hours, or overnight. Drying is accomplished in a fume hood, forced air convection oven or vacuum oven.

Said “evaporating” is accomplished by using a conventional method in the field. Slow evaporation is accomplished in a container covered by sealing film with pinholes. Fast evaporation is accomplished in an open container.

Said “cooling” is accomplished by using conventional methods in the field such as slow cooling and rapid cooling. Slow cooling is usually accomplished at the speed of 0.1° C./min. Rapid cooling is usually accomplished by transferring the sample directly from environment which is no lower than room temperature to refrigerator for cooling.

In the present disclosure, “crystal” or “crystalline form” refers to the crystal or the crystalline form being identified by the X-ray diffraction pattern shown herein. Those skilled in the art are able to understand that physicochemical properties discussed herein can be characterized. The experimental errors depend on the instrument conditions, the sample preparation and the purity of samples. In particular, those skilled in the art generally know that the X-ray diffraction pattern typically varies with the experimental conditions. It is necessary to point out that, the relative intensity of the diffraction peaks in the X-ray diffraction pattern may also vary with the experimental conditions; therefore, the order of the diffraction peak intensities cannot be regarded as the sole or decisive factor. In fact, the relative intensity of the diffraction peaks in the X-ray powder diffraction pattern is related to the preferred orientation of the crystals, and the diffraction peak intensities shown herein are illustrative and identical diffraction peak intensities are not required. In addition, the experimental error of the diffraction peak position is usually 5% or less, and the error of these positions should also be considered. An error of ±0.2° is usually allowed. In addition, due to experimental factors such as sample thickness, the overall offset of the diffraction peak is caused, and a certain offset is usually allowed. Thus, it will be understood by those skilled in the art that a crystalline form of the present disclosure is not necessarily to have the exact same X-ray diffraction pattern of the example shown herein. Any crystalline forms whose X-ray diffraction patterns have the same or similar characteristic peaks should be within the scope of the present disclosure. Those skilled in the art can compare the patterns shown in the present disclosure with that of an unknown crystalline form in order to identify whether these two groups of patterns reflect the same or different crystalline forms. In some embodiments, crystalline Form CSI and Form CSIII of the present disclosure is pure and substantially free of any other crystalline forms. In the present disclosure, the term “substantially free” when used to describe a novel crystalline form, it means that the content of other crystalline forms in the novel crystalline form is less than 20% (w/w), specifically less than 10% (w/w), more specifically less than 5% (w/w) and further more specifically less than 1% (w/w).

In the present disclosure, the term “about” when referring to a measurable value such as weight, time, temperature, and the like, is meant to encompass variations of ±10%, ±5%, ±1%, ±0.5%, or even ±0.1% of the specified amount.

### BRIEF DESCRIPTION OF THE DRAWINGS

FIG. 1 shows an XRPD pattern of Form CSI in Example 1.

FIG. 2 shows a TGA curve of Form CSI in Example 1.

FIG. 3 shows a DSC curve of Form CSI in Example 1.

FIG. 4 shows a ¹H NMR spectrum of Form CSI in Example 1.

FIG. 5 shows an XRPD pattern of Form CSI in Example 2.

FIG. 6 shows an XRPD pattern of Form CSIII in Example 3.

FIG. 7 shows an XRPD pattern of Form CSIII in Example 4.

FIG. 8 shows an XRPD pattern of Form M2 in Example 10.

FIG. 9 shows a PSD diagram of Form M2 in Example 10.

FIG. 10 shows a DVS plot of Form M2 in Example 10.

FIG. 11 shows an XRPD pattern of Form M2 in Example 11.

FIG. 12 shows an XRPD pattern of Form M2 in Example 12.

FIG. 13 shows an XRPD pattern of Form M2 in Example 14.

FIG. 14 shows an XRPD pattern of the solid obtained after 2-hour stirring in the comparative example.

FIG. 15 shows an XRPD pattern of the solid obtained after 30-hour stirring in the comparative example.

### DETAILED DESCRIPTION

The present disclosure is further illustrated by the following examples which describe the preparation and use of the crystalline forms of the present disclosure in detail. It is obvious to those skilled in the art that many changes in the materials and methods can be accomplished without departing from the scope of the present disclosure.

The abbreviations used in the present disclosure are explained as follows:

XRPD: X-ray Powder Diffraction

DSC: Differential Scanning calorimetry

TGA: Thermo Gravimetric Analysis

DVS: Dynamic Vapor Sorption

¹H NMR: Proton Nuclear Magnetic Resonance

PSD: Particle Size Distribution

HPLC: High Performance Liquid Chromatography

Instruments and methods used for data collection:

X-ray powder diffraction patterns in Example 1-4, 10-11, 13-14 and comparative examples of the present disclosure were acquired by a Bruker D2 PHASER X-ray powder diffractometer. The parameters of the X-ray powder diffraction method of the present disclosure are as follows:
X-Ray Reflection: Cu, Kα
Kα1 (Å): 1.54060; Kα2 (Å): 1.54439
Kα2/Kα1 intensity ratio: 0.50
Voltage: 30 (kV)
Current: 10 (mA)
Scan range: from 3.0 degree to 40.0 degree

X-ray powder diffraction patterns in Example 12 of the present disclosure were acquired by a Bruker D8 Discover X-Ray powder diffractometer. The parameters of the X-ray powder diffraction method of the present disclosure are as follows:
X-Ray Reflection: Cu, Kα
Kα1 (Å): 1.54060; Kα2 (Å): 1.54439
Kα2/Kα1 intensity ratio: 0.50
Voltage: 40 (kV)
Current: 40 (mA)
Scan range: from 4.0 degree to 40.0 degree

Differential scanning calorimetry (DSC) data in the present disclosure were acquired by a TA Q2000. The parameters of the DSC method of the present disclosure were as follows:
Heating rate: 10° C./min
Purge gas: nitrogen

Thermo gravimetric analysis (TGA) data in the present disclosure were acquired by a TA Q500. The parameters of the TGA method of the present disclosure were as follows:
Heating rate: 10° C./min
Purge gas: nitrogen

Proton nuclear magnetic resonance spectrum data (¹H NMR) were collected from a Bruker Avance II DMX 400M HZ NMR spectrometer. 1-5 mg of sample was weighed and dissolved in 0.5 mL of deuterated dimethyl sulfoxide to obtain a solution with a concentration of 2-10 mg/mL.

High Performance Liquid Chromatography (HPLC) data in the present disclosure were collected from an Agilent 1260 with Variable Wavelength Detector (VWD).

The HPLC method parameters for solubility test in the present disclosure are as follows:
1. Column: Waters XBridge C18 150×4.6mm, 5 μm
2. Mobile Phase: A: 0.1% TFA in H₂O
   B: 0.1% TFA in Acetonitrile
Gradient:

| Time (min) | % B |
|---|---|
| 0.0 | 10 |
| 1.0 | 10 |
| 17.0 | 80 |
| 20.0 | 80 |
| 20.1 | 10 |
| 25.0 | 10 |

3. Flow rate: 1 mL/min
4. Injection Volume: 5 μL
5. Detection wavelength: 250 nm
6. Column Temperature: 40° C.
7. Diluent: Acetonitrile/H₂O (9:1, v/v)

The particle size distribution data in the present disclosure were acquired by an S3500 laser particle size analyzer of Microtrac. Microtrac S3500 is equipped with an SDC (Sample Delivery Controller). The test is carried out in wet mode, and the dispersion medium is Isopar G. The parameters are as follows:

| | |
|---|---|
| Size distribution: Volume | Run Time: 10 s |
| Dispersion medium: Isopar G | Particle coordinates: Standard |
| Run Number: 3 times | Fluid refractive index: 1.42 |
| Particle Transparency: Trans | Residuals: Enabled |
| Particle refractive index: 1.59 | Flow rate: 60* |
| Particle shape: Irregular | Filtration: Enabled |
| Ultrasonication power: 30 W | Ultrasonication time: 0 s |

*Flow rate 60% is 60% of 65 mL/s.

Dynamic Vapor Sorption (DVS) is measured via an SMS (Surface Measurement Systems Ltd.) intrinsic DVS instrument. Its control software is DVS-Intrinsic control software, and its control software is DVS-Intrinsic control software. Typical Parameters for DVS test are as follows:
Temperature: 25° C.
Gas and flow rate: N₂, 200 mL/min
dm/dt=0.002
RH range: 0% RH to 95% RH

Unless otherwise specified, the following examples were conducted at room temperature. Said “room temperature” is not a specific temperature, but a temperature range of 10-30° C.

According to the present disclosure, cabozantinib and/or its salt used as a raw material is solid (crystalline and amorphous), oil, liquid form or solution. Preferably, Compound I and/or its salt used as a raw material is a solid.

Raw materials of cabozantinib and/or a salt thereof used in the following examples were prepared by known methods in the prior art, for example, the method disclosed in CN102388024A.

### Example 1 Preparation of Form CSI

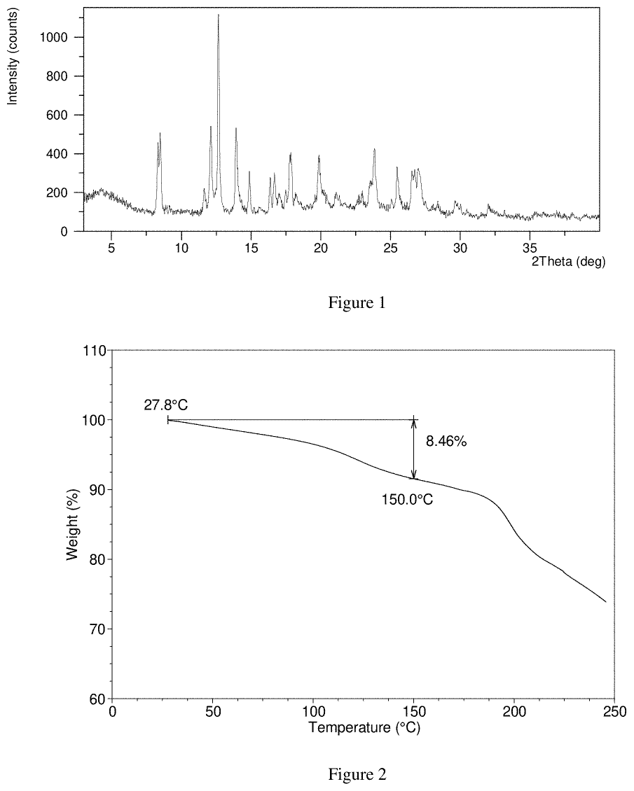

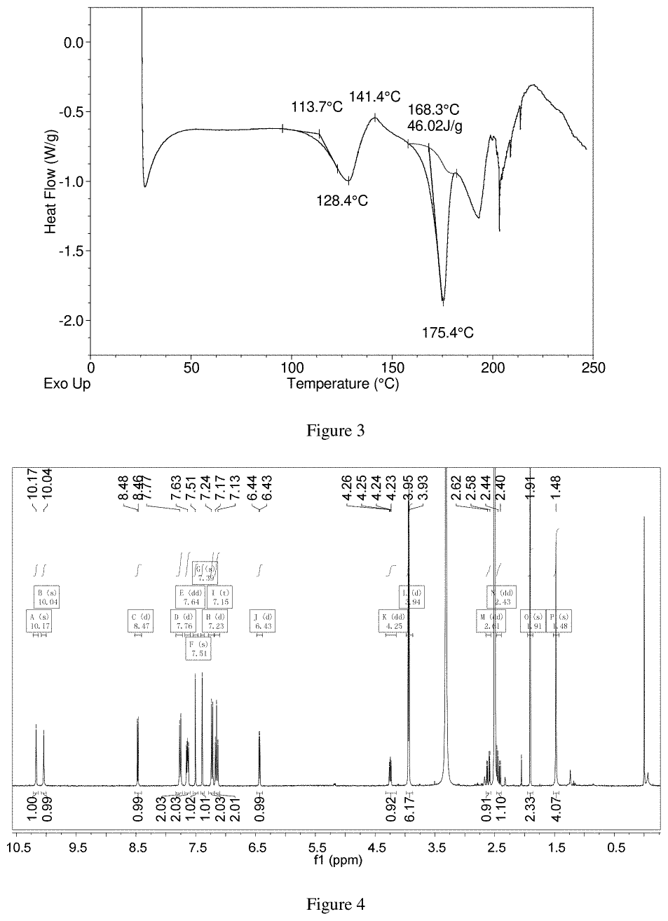

100.5 mg of Compound I was weighed, and mixed with 10 mL of acetic acid/toluene (1:1, v/v) solvent mixture. The mixture was magnetically stirred at 50° C. until the solid was completely dissolved. The resulting clear solution was left to stand at 50° C. to evaporate, and a solid sample was obtained after about 15 days.
The obtained solid was characterized by XRPD, TGA, DSC and ¹H NMR. The XRPD pattern is substantially as depicted in FIG. 1, and the XRPD data are listed in Table 1.
The characterization results of TGA, DSC and ¹H NMR are summarized as follows: The TGA curve is substantially as depicted in FIG. 2. It shows about 8.5% weight loss after heating to 150° C., corresponding to the loss of acetic acid in the heating process. Form CSI is an acetic acid solvate.
The DSC curve is substantially as depicted in FIG. 3, which shows an endothermic peak at around 114° C., an exothermic peak at around 141° C., and an endothermic peak at around 168° C.
The ¹H NMR spectrum of Form CSI is substantially as depicted in FIG. 4. The chemical shift results are consistent with the structure of the compound (C₂₈H₂₄FN₃O₅·C₄H₆O₅). The three active hydrogen atoms of malic acid is not observed, and the peak at the chemical shift of 1.91 corresponds to the inactive hydrogen of acetic acid. The detailed data are:¹H NMR (400 MHz, DMSO-d6) δ 10.17 (s, 1H), 10.04 (s, 1H), 8.47 (d, J=5.2 Hz, 1H), 7.76 (d, J=8.9 Hz, 2H), 7.64 (dd, J=9.1, 5.1 Hz, 2H), 7.51 (s, 1H), 7.39 (s, 1H), 7.23 (d, J=9.0 Hz, 2H), 7.15 (t, J=8.9 Hz, 2H), 6.43 (d, J=5.2 Hz, 1H), 4.25 (dd, J=7.7, 5.0 Hz, 1H), 3.94 (d, J=5.2 Hz, 6H), 2.61 (dd, J=15.7, 5.0 Hz, 1H), 2.43 (dd, J=15.7, 7.7 Hz, 1H), 1.91 (s, 2H), 1.48 (s, 4H).

TABLE 1

| 2 Theta | d Spacing | Relative intensity % |
|---|---|---|
| 8.32 | 10.63 | 34.68 |
| 8.48 | 10.43 | 39.04 |
| 11.65 | 7.60 | 11.38 |
| 12.11 | 7.31 | 43.70 |
| 12.65 | 7.00 | 100.00 |
| 13.92 | 6.36 | 42.79 |
| 14.88 | 5.95 | 20.65 |
| 16.36 | 5.42 | 18.36 |
| 16.67 | 5.32 | 20.44 |
| 17.89 | 4.96 | 25.60 |
| 19.85 | 4.47 | 29.93 |
| 21.16 | 4.20 | 9.05 |
| 23.83 | 3.73 | 33.28 |
| 25.47 | 3.50 | 24.33 |
| 26.54 | 3.36 | 22.29 |
| 27.04 | 3.30 | 22.31 |
| 28.25 | 3.16 | 4.16 |
| 29.70 | 3.01 | 6.08 |
| 32.06 | 2.79 | 4.80 |

### Example 2 Preparation of Form CSI

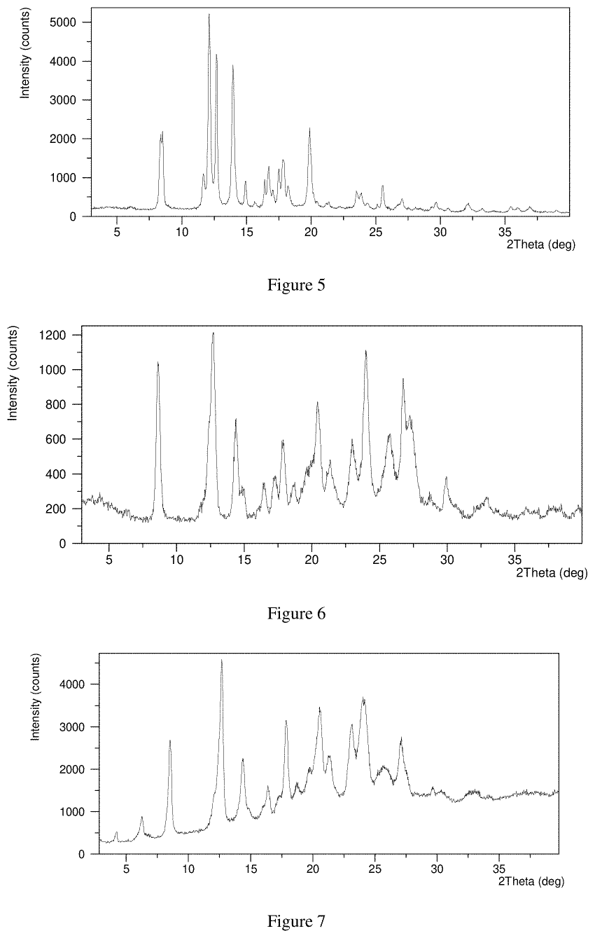

2033.1 mg of compound I was weighed, and mixed with 6.0 mL of acetic acid. The mixture was stirred magnetically at 50° C. until the solid was completely dissolved. The solution was cooled to room temperature naturally and filtered to obtain a clear solution. A total of 20.0 mL of toluene was added in portions (1.0 mL each time) to the clear solution with stirring. The resulting suspension was transferred to an environment at 5° C. and stirred for about 24 h. The precipitated solid was separated.
The obtained solid was characterized to be Form CSI by XRPD. The XRPD pattern and XRPD data are shown in FIG. 5 and Table 2, respectively.

TABLE 2

| 2 Theta | d Spacing | Relative Intensity % |
|---|---|---|
| 8.31 | 10.64 | 38.02 |
| 8.47 | 10.44 | 39.75 |
| 11.63 | 7.61 | 18.09 |
| 12.08 | 7.33 | 100.00 |
| 12.65 | 7.00 | 79.41 |
| 13.92 | 6.36 | 73.94 |
| 14.88 | 5.95 | 14.14 |
| 15.61 | 5.68 | 3.84 |
| 16.36 | 5.42 | 15.49 |
| 16.67 | 5.32 | 22.24 |
| 17.00 | 5.22 | 9.61 |
| 17.47 | 5.08 | 19.47 |
| 17.79 | 4.98 | 25.15 |
| 17.88 | 4.97 | 24.06 |
| 18.17 | 4.88 | 11.89 |
| 19.85 | 4.47 | 41.93 |
| 21.32 | 4.16 | 4.34 |
| 23.47 | 3.79 | 9.51 |
| 23.83 | 3.73 | 9.04 |
| 24.31 | 3.66 | 3.77 |
| 25.08 | 3.55 | 3.52 |
| 25.48 | 3.49 | 13.01 |
| 26.99 | 3.30 | 6.33 |
| 29.63 | 3.01 | 4.49 |
| 32.09 | 2.79 | 4.09 |

### Example 3 Preparation of Form CSIII

5.1 g of compound I was weighed and dissolved in 25.0 mL of acetic acid, and then stirred at 100° C. until the solid was completely dissolved. 25.0 mL of toluene was added after the solution was cooled to room temperature. The solution was filtered at room temperature, transferred to a reactor and then cooled to 0° C. 52.1 mg of seed crystals were added and the mixture was aged with mechanical stirring for 1.5 hours. Then, 50.0 mL of isopropyl acetate was added, and the solid was separated after stirring for 20 hours. The obtained solid was stirred in 100.5 mL of toluene/water (200:1, v/v) for about 2 minutes, and then the solid was separated.
The obtained solid was characterized to be Form CSIII by XRPD. The XRPD pattern is substantially as depicted in FIG. 6, and the XRPD data are listed in Table 3.

TABLE 3

| 2 Theta | d Spacing | Relative Intensity % |
|---|---|---|
| 8.57 | 10.32 | 80.15 |
| 12.68 | 6.98 | 100.00 |
| 14.37 | 6.16 | 52.82 |
| 14.90 | 5.95 | 15.34 |
| 16.44 | 5.39 | 18.43 |
| 17.24 | 5.14 | 21.60 |
| 17.86 | 4.97 | 40.50 |
| 18.67 | 4.75 | 17.60 |
| 20.43 | 4.35 | 62.33 |
| 21.34 | 4.16 | 30.84 |
| 22.99 | 3.87 | 40.83 |
| 24.00 | 3.71 | 88.06 |
| 25.75 | 3.46 | 45.21 |
| 26.76 | 3.33 | 73.15 |
| 27.40 | 3.25 | 48.68 |
| 28.74 | 3.11 | 10.75 |
| 29.92 | 2.99 | 21.38 |
| 32.95 | 2.72 | 10.42 |
| 35.85 | 2.50 | 3.74 |

### Example 4 Preparation of Form CSIII

493.1 mg of compound I was weighed and dissolved in 1.5 mL of acetic acid. The solution was stirred magnetically at 80° C. until the solid was completely dissolved. The solution was filtered after naturally cooled to room temperature. A total of 5.0 mL of toluene was added to the clear solution with stirring and then transferred to an environment at 5° C. After stirring for about 15 hours, the solid was separated by filtration and transferred to 60° C./75% RH (relative humidity) conditions and left overnight.
The obtained solid was placed on a variable-temperature stage, and the stage was placed in a sealed cavity chamber. The solid was heated to 100° C. and then cooled to 30° C. with nitrogen purging to obtain a white crystalline solid.
The obtained solid was characterized to be Form CSIII by XRPD. The XRPD pattern is substantially as depicted in FIG. 7, and the XRPD data are listed in Table 4.

TABLE 4

| 2 Theta | d Spacing | Relative Intensity % |
|---|---|---|
| 4.22 | 20.93 | 5.98 |
| 6.28 | 14.08 | 13.11 |
| 8.53 | 10.37 | 56.71 |
| 12.68 | 6.98 | 100.00 |
| 14.37 | 6.16 | 39.31 |
| 16.39 | 5.41 | 21.73 |
| 17.20 | 5.15 | 14.08 |
| 17.86 | 4.97 | 59.77 |
| 17.96 | 4.94 | 50.93 |
| 18.74 | 4.74 | 21.46 |
| 19.66 | 4.52 | 28.03 |
| 20.57 | 4.32 | 64.19 |
| 21.30 | 4.17 | 35.73 |
| 23.13 | 3.84 | 53.35 |
| 24.01 | 3.71 | 66.94 |
| 24.21 | 3.68 | 64.51 |
| 25.76 | 3.46 | 25.61 |
| 27.09 | 3.29 | 40.08 |
| 29.65 | 3.01 | 10.62 |
| 30.46 | 2.93 | 7.42 |
| 32.53 | 2.75 | 5.43 |
| 33.20 | 2.70 | 5.33 |

### Example 5 Kinetic Solubility Study

Simulated gastrointestinal fluids such as FaSSIF (Fasted state simulated intestinal fluids) and FeSSIF (Fed state simulated intestinal fluids) are biorelevant media. Solubility in such media is close to that in human environment because these media can reflect the effects of gastrointestinal environment on drug release better.
20 mg of Form CSI and 20 mg of crystalline forms in the prior arts were suspended into 1.5 mL of water to get saturated solutions. After equilibrated for 1 h and 4 h, concentrations (mg/mL) of the saturated solutions were measured by HPLC. The results are listed in Table 5.

TABLE 5

| | Solid form | Time | H₂O |
|---|---|---|---|
| Solubility | Form CSI | 1 h | 0.35 |
| (mg/mL) | | 4 h | 0.36 |
| | Form M4 | 1 h | 0.072 |
| | | 4 h | 0.0028 |
| | Form N-2 | 1 h | 0.15 |
| | | 4 h | 0.15 |

20 mg of Form CSIII and 20 mg of crystalline forms in the prior arts were suspended into 1.5 mL of SGF, 1.5 mL of FaSSIF, 1.5 mL of FeSSIF and 1.5 mL of water to get saturated solutions. After equilibrated for 1 h and 4 h, concentrations (mg/mL) of the saturated solutions were measured by HPLC. The results are listed in Table 6.

TABLE 6

| | Solid form | Time | FaSSIF | FeSSIF | H₂O |
|---|---|---|---|---|---|
| Solubility | Form CSIII | 1 h | 0.025 | 0.13 | 0.30 |
| (mg/mL) | | 4 h | 0.0083 | 0.12 | 0.24 |
| | FormM4 | 1 h | 0.012 | 0.062 | 0.072 |
| | | 4 h | 0.0019 | 0.065 | 0.0028 |
| | FormN-2 | 1 h | 0.011 | 0.047 | 0.15 |
| | | 4 h | 0.0079 | 0.043 | 0.15 |

The results show that the solubility of Form CSI and CSIII of the present disclosure are higher than that of prior art.

### Example 6 Purification Effect of Form CSI

Form CSI was prepared from the starting material. HPLC was applied to test the chemical purity of the starting material and Form CSI. The results show that the chemical purity of Form CSI is obviously improved by crystallization from the starting material, and the contents of all impurity are reduced. This indicated that Form CSI in the present invention has good purification effect.

### Example 7 Flowability of Form CSI

Compressibility index or Carr Index is usually utilized to evaluate the flowability of powder or granules during the drug product process. Compressibility index test method is as follows: a certain amount of powder was added into a measuring cylinder and bulk volume was recorded. Then the powder was tapped to make it in the tightest state and the tapped volume was recorded. The bulk density (ρ₀), tapped density (ρ_{f}) were calculated and compressibility index was calculated according to c=(ρ_{f}−ρ₀)/ρ_{f}.
Criteria of flowability according to United States Pharmacopoeia USP1174, which is shown in Table 7.

TABLE 7

| Compressibility index (%) | Flowability |
|---|---|
| ≤10 | Excellent |
| 11-15 | Good |
| 16-20 | Fair |
| 21-25 | Passable |
| 26-31 | poor |
| 32-37 | Very poor |
| >38 | Very, very poor |

Flowability evaluation results of Form CSIII and prior art are presented in Table 8, which indicate that the flowability of Form CSIII is remarkably superior to that of prior art.

TABLE 8

| | Bulk density | Tapped density | Compressibility |
|---|---|---|---|
| Solid form | (g/ml) | (g/ml) | index |
| Form CSIII | 0.319 | 0.376 | 15% |
| Form N-2 | 0.194 | 0.256 | 24% |
| Form M4 | 0.251 | 0.387 | 35% |

### Example 8 Compressibility of CS1

Manual tablet press was used for compression. 80 mg of Form CSIII and crystalline forms in the prior art were weighed and added into the dies of a φ6 mm round tooling, respectively, compressed at 10 KN manually, then stored at room temperature for 24 h until complete elastic recovery. Hardness (H) was tested with an intelligent tablet hardness tester. Diameter (D) and thickness (L) were tested with caliper. Tensile strength of the powder was calculated with the following formula: T=2H/πDL. Under a certain force, the greater the tensile strength, the better the compressibility.
Three tests on each sample were performed to calculate the tensile strength of the powder and the average value. The results are presented in Table 9.

TABLE 9

| Solid Form | Form CSIII | Form N-2 | Form M4 |
|---|---|---|---|
| Average tensile | 1.56 | 1.02 | 1.23 |
| strength (MPa) | | | |

The results show that Form CSIII has better compressibility compared with than prior art.

### Example 9 Adhesiveness of Form CSI

30 mg of Form CSIII and crystalline forms in the prior art were weighed separately and then added into the dies of φ8 mm round tooling, compressed at 10 KN and held for 30 s. The punch was weighed and amount of material sticking to the punch was calculated. The compression was repeated twice and the cumulative amount, maximum amount and average amount of material sticking to the punch during the compression were recorded. Detailed experimental results are shown in Table 10.

TABLE 10

| Solid Form | Maximum amount (mg) | Average amount (mg) |
|---|---|---|
| Form CSIII | 0.06 | 0.045 |
| Form N-2 | 0.26 | 0.20 |
| Form M4 | 0.11 | 0.08 |

Test results indicate that the maximum amount sticking to the punch of the prior art is over twice of that of Form CSIII. The adhesiveness of CS III is superior to the prior art form.

### Example 10 Preparation of Form M2

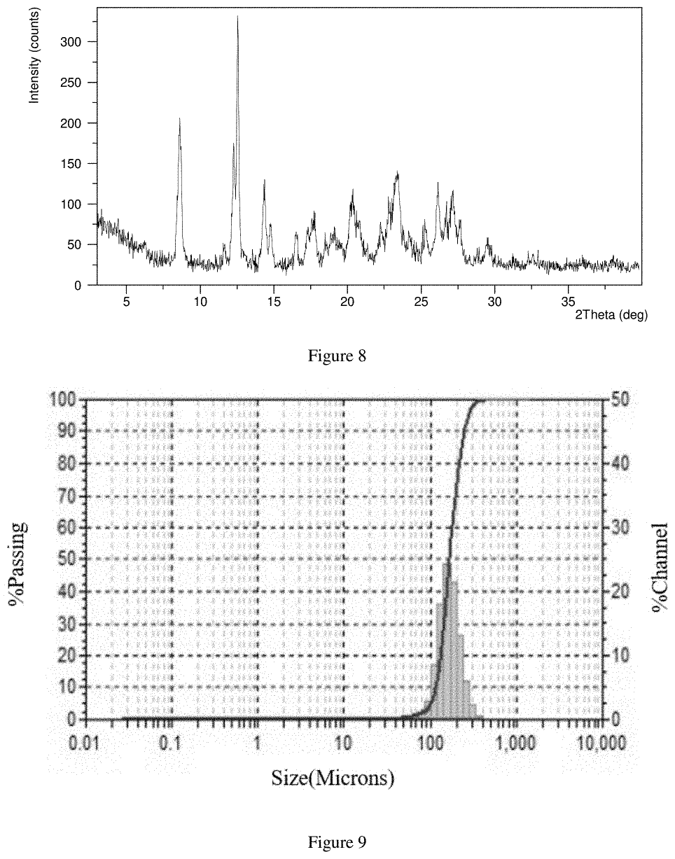

100.11 g of compound I was weighed into a 1-L glass container, and a mixed solvent of acetic acid and toluene was added to dissolve the solid and obtain a clear solution. The clear solution was filtered into a 5-L reactor and cooled to 5-15° C. 2.02 g of Form M2 seed crystals were added to the system and the system was aged for 0.5 h. Isopropyl acetate and toluene were added slowly into the suspension. After a solid was precipitated, the solid was separated by suction filtration. The solid was dried in a forded air convection oven at 40° C. (Humidity: 30% RH-40% RH). Characterization shows that the obtained solid is Form M2. The XRPD pattern and data are shown in FIG. 8 and Table 11, respectively.

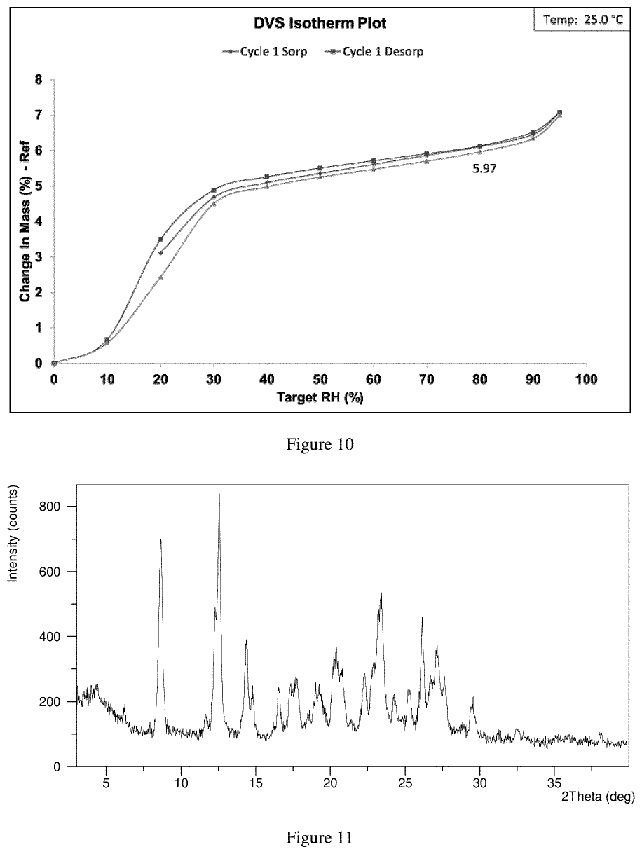

The obtained solid was test, and the chemical purity of the obtained solid was 99.77%. The solvent residues of acetic acid, toluene, isopropyl acetate, and n-heptane were less than 1250 ppm, 325 ppm, 756 ppm, and 2324 ppm, respectively, which meets the requirements of ICH. The particle size distribution diagram of the solid is shown in FIG. 9. The diagram basically presents a normal distribution with D90 of 244.3 μm, which indicates that the particle size is relatively uniform. Larger particle size facilitates the filtration and separation processes. The Dynamic Vapor Sorption (DVS) of the obtained solid is shown in FIG. 10. When the humidity is lower than 30% RH, Form M2 dehydrated rapidly and the lattice water may be removed. Therefore, the drying humidity should be kept above 30% RH.

TABLE 11

| 2 Theta | d Spacing | Relative Intensity % |
|---|---|---|
| 6.26 | 14.12 | 4.69 |
| 8.60 | 10.28 | 57.00 |
| 11.64 | 7.60 | 8.35 |
| 12.27 | 7.21 | 51.62 |
| 12.56 | 7.05 | 100.00 |
| 14.35 | 6.17 | 31.84 |
| 14.78 | 6.00 | 17.42 |
| 16.46 | 5.38 | 12.50 |
| 17.29 | 5.13 | 14.37 |
| 17.77 | 4.99 | 19.28 |
| 18.49 | 4.80 | 10.17 |
| 19.00 | 4.67 | 11.71 |
| 20.29 | 4.38 | 25.55 |
| 22.26 | 3.99 | 14.80 |
| 23.20 | 3.83 | 32.44 |
| 23.45 | 3.79 | 37.59 |
| 24.20 | 3.68 | 12.52 |
| 25.31 | 3.52 | 15.82 |
| 26.12 | 3.41 | 31.65 |
| 26.68 | 3.34 | 20.13 |
| 27.16 | 3.28 | 30.57 |
| 27.64 | 3.23 | 17.65 |
| 28.82 | 3.10 | 4.45 |
| 29.47 | 3.03 | 8.83 |

### Example 11 Preparation of Form M2

5.06 g of compound I was weighed into a 100-mL glass vial, and a mixed solvent of acetic acid and toluene was added to dissolve the solid and obtain a clear solution. The solution was filtered into a 250-mL reactor and cooled to 0-5° C. 52.1 mg of Form M2 seed crystals were added to the system and the system was aged for 0.5 h. Isopropyl acetate was added slowly into the suspension. After a solid was precipitated, the solid was separated by suction filtration. The solid was dried by a forded air convection oven at 30° C. (the humidity was no less than 40% RH). Characterization shows that the obtained solid is Form M2. The XRPD pattern and data are shown in FIG. 11 and Table 12, respectively. The chemical purity of the obtained solid was 99.77%. The solvent residue of acetic acid was less than 1250 ppm.

TABLE 12

| 2 Theta | d Spacing | Relative Intensity % |
|---|---|---|
| 3.99 | 22.13 | 23.45 |
| 4.33 | 20.39 | 22.26 |
| 6.21 | 14.24 | 13.19 |
| 8.65 | 10.22 | 81.76 |
| 11.62 | 7.62 | 10.30 |
| 12.25 | 7.23 | 46.01 |
| 12.56 | 7.05 | 100.00 |
| 14.36 | 6.16 | 41.53 |
| 14.43 | 6.15 | 36.67 |
| 14.79 | 5.98 | 22.32 |
| 16.51 | 5.37 | 21.89 |
| 17.26 | 5.13 | 20.25 |
| 17.77 | 4.99 | 25.52 |
| 18.53 | 4.78 | 11.25 |
| 19.00 | 4.67 | 21.70 |
| 20.25 | 4.38 | 34.50 |
| 20.39 | 4.35 | 36.45 |
| 20.79 | 4.27 | 28.62 |
| 22.31 | 3.98 | 26.62 |
| 22.79 | 3.90 | 27.36 |
| 23.24 | 3.82 | 50.11 |
| 23.45 | 3.79 | 53.57 |
| 24.22 | 3.67 | 17.88 |
| 25.24 | 3.53 | 19.28 |
| 26.15 | 3.41 | 51.00 |
| 26.68 | 3.34 | 26.73 |
| 27.10 | 3.29 | 36.81 |
| 27.64 | 3.22 | 25.65 |
| 28.86 | 3.09 | 7.66 |
| 29.37 | 3.04 | 13.66 |
| 29.56 | 3.02 | 15.73 |
| 32.52 | 2.75 | 5.55 |

### Example 12 Preparation of Form M2

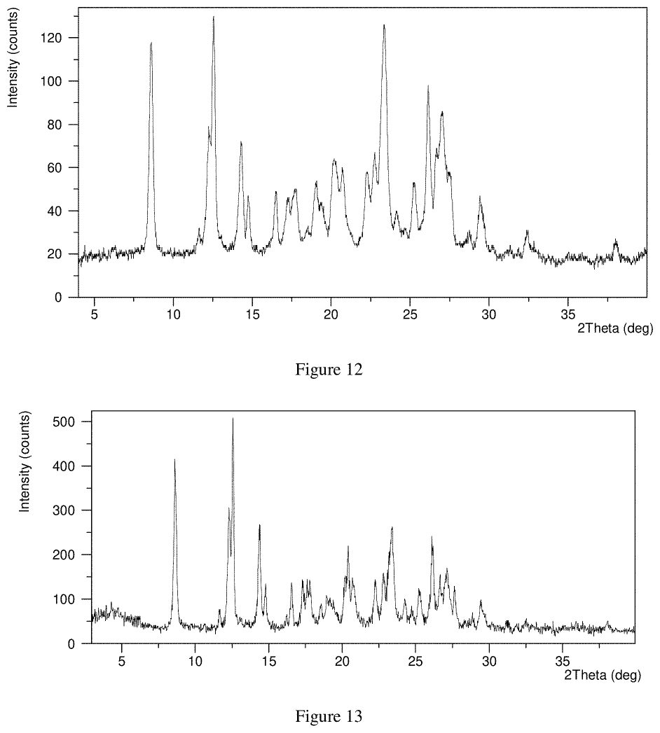

About 8.00 g of cabozantinib freebase and 2.25 g of (S)-malic acid was weighed in a 100-mL glass vial, and a mixed solvent of acetic acid and toluene was added to dissolve the solid and obtain a clear solution. The solution was filtered into a 500-mL reactor and cooled to −5-15° C. About 200 mg of Form M2 seed crystals were added to the system. Isopropyl acetate and toluene were added slowly into the system. After a solid was precipitated, the solid was separated by suction filtration. The solid was dried by a forded air convection oven at 40° C. (the humidity was no less than 40% RH). And then Form M2 was obtained. The XRPD pattern and XRPD data are shown in FIG. 12 and Table 13, respectively.

TABLE 13

| 2 Theta | d Spacing | Relative Intensity % |
|---|---|---|
| 8.59 | 10.29 | 87.97 |
| 11.63 | 7.61 | 9.89 |
| 12.25 | 7.23 | 52.46 |
| 12.56 | 7.05 | 100.00 |
| 14.33 | 6.18 | 46.23 |
| 14.74 | 6.01 | 24.69 |
| 16.53 | 5.36 | 27.07 |
| 17.25 | 5.14 | 23.46 |
| 17.80 | 4.98 | 27.67 |
| 19.04 | 4.66 | 30.48 |
| 20.11 | 4.42 | 38.66 |
| 20.70 | 4.29 | 37.18 |
| 22.21 | 4.00 | 34.76 |
| 22.76 | 3.91 | 43.97 |
| 23.40 | 3.80 | 94.66 |
| 24.14 | 3.69 | 19.47 |
| 25.26 | 3.53 | 31.11 |
| 26.15 | 3.41 | 70.48 |
| 26.63 | 3.35 | 43.46 |
| 27.03 | 3.30 | 61.29 |
| 27.58 | 3.23 | 34.06 |
| 28.80 | 3.10 | 10.00 |
| 29.41 | 3.04 | 26.61 |
| 31.28 | 2.86 | 4.36 |
| 32.47 | 2.76 | 11.40 |
| 38.07 | 2.36 | 7.47 |

### Example 13 Preparation of Form M2

About 50 mg of Compound I was weighed into a 10-mL glass bottle, and a mixed solvent of acetic acid and toluene was added to dissolve the solid and obtain a clear solution. The solution was filtered into a 20-mL reactor and cooled to 5° C. N-Propanol, isopropanol, methyl isobutyl ketone, ethyl acetate or isopropyl acetate was added slowly to the system as anti-solvent. After a solid was precipitated, the solid was separated by suction filtration. The solid was dried by blast oven at 40° C. (the humidity was no less than 40% RH) to obtain Form M2.

### Example 14 Preparation of Form M2

About 493.1 mg of Compound I was weighed into a 5-mL glass vial, and 1.5 mL of acetic acid was added. The mixture was heated to 80° C. to obtain a clear solution. The obtained solution was cooled to room temperature and filtered into a 20-mL glass vial. 5.0 mL of toluene was added to the solution, and then transferred to 5° C. and stirred overnight. The precipitated solid was filtered and dried under 60° C./75% RH condition for 22 h to obtain Form M2. The XRPD pattern and XRPD data are shown in FIG. 13 and Table 14, respectively.

TABLE 14

| 2 Theta | d Spacing | Relative Intensity % |
|---|---|---|
| 8.61 | 10.27 | 80.56 |
| 8.72 | 10.14 | 47.82 |
| 11.64 | 7.60 | 9.23 |
| 12.28 | 7.21 | 53.35 |
| 12.56 | 7.05 | 100.00 |
| 14.37 | 6.16 | 48.00 |
| 14.81 | 5.98 | 17.80 |
| 16.55 | 5.36 | 22.33 |
| 17.30 | 5.12 | 19.25 |
| 17.61 | 5.04 | 20.81 |
| 17.83 | 4.98 | 20.20 |
| 18.55 | 4.78 | 11.40 |
| 18.95 | 4.68 | 15.69 |
| 20.19 | 4.40 | 24.42 |
| 20.42 | 4.35 | 35.53 |
| 20.72 | 4.29 | 22.68 |
| 22.29 | 3.99 | 22.95 |
| 22.76 | 3.91 | 23.73 |
| 23.39 | 3.80 | 47.60 |
| 24.25 | 3.67 | 13.82 |
| 24.77 | 3.59 | 7.83 |
| 25.24 | 3.53 | 18.39 |
| 26.08 | 3.41 | 31.18 |
| 26.19 | 3.40 | 38.94 |
| 26.67 | 3.34 | 26.38 |
| 27.11 | 3.29 | 25.88 |
| 27.64 | 3.23 | 18.37 |
| 29.42 | 3.04 | 12.93 |

### Comparative Example: Preparation of Form M2 Disclosed in WO2015177758A1

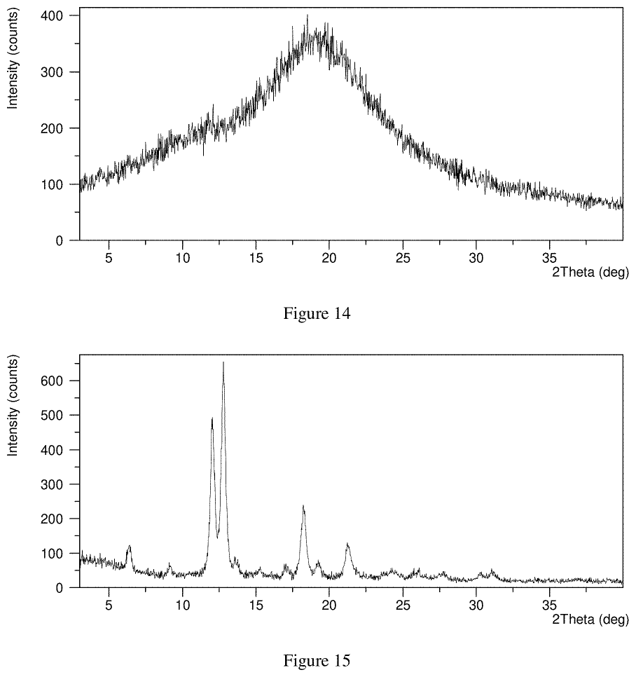

335.4 mg of compound I was weighed into a 20-mL glass vial, and 1 mL of propionic acid was added. A clear solution was obtained by heating. The solution was cooled to room temperature and 10 mL of methyl-tert-butyl ether was added with stirring. After aging for 2 h, the solid was sampled and characterized to be amorphous, and the XRPD pattern is shown in FIG. 14. After stirred for about another 30 h, the solid was separated and characterized to be Form N-1, and the XRPD pattern is shown in FIG. 15.
The experimental process shows that the preparation method of Form M2 disclosed in the prior art is not controllable and Form M2 can't be obtained repeatedly.
The examples described above are only for illustrating the technical concepts and features of the present disclosure, and intended to make those skilled in the art being able to understand the present disclosure and thereby implement it, and should not be concluded to limit the protective scope of this disclosure. Any equivalent variations or modifications according to the spirit of the present disclosure should be covered by the protective scope of the present disclosure.

1. A process for preparing crystalline form M2 of Compound I, wherein the process comprises: dissolving Compound I solid or a mixed solid of cabozantinib and (S)-malic acid in a solvent and then adding an anti-solvent to precipitate a solid, then drying the obtained solid under a condition of above 30% relative humidity (RH) to obtain crystalline form M2, wherein said solvent is an organic acid or a mixture of organic acid and aromatic hydrocarbon; said anti-solvent is an aromatic hydrocarbon or an ester or an alcohol or a ketone or a mixture of an aromatic hydrocarbon and an ester or a mixture of an aromatic hydrocarbon and a ketone; wherein the X-ray powder diffraction pattern of said crystalline form M2 comprises characteristic peaks at 2 theta values of 8.6°±0.2 °, 12.6 °±0.2°, 20.2°±0.2°, 23.4°±0.2°, and 26.1°±0.2.

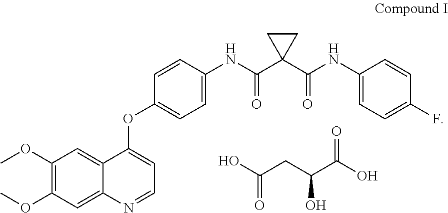

2. The process for preparing crystalline form M2 of Compound I according to claim 1, wherein said organic acid is acetic acid, said aromatic hydrocarbon is toluene, said ester is ethyl acetate or isopropyl acetate, said ketone is methyl isobutyl ketone, and said alcohol isopropanol or n-propanol.

3. The process for preparing crystalline form M2 of Compound I according to claim 1, wherein the temperature of the solvent system is below 15° C. when adding an anti-solvent.

4. The process for preparing crystalline form M2 of Compound I according to claim 3, wherein the temperature of the solvent system is −5° C. to 10° C. when adding an anti-solvent.

5. The process for preparing crystalline form M2 of Compound I according to claim 1, wherein seed crystals of crystalline form M2 can be added before adding the anti-solvent and the amount of the seed crystals are 1 wt % to 10 wt %.

6. The process for preparing crystalline form M2 of Compound I according to claim 1, wherein the volume ratio of said solvent and said anti-solvent is 1:1 to 1:10.

7. The process for preparing crystalline form M2 of Compound I according to claim 6, wherein the volume ratio of said solvent and said anti-solvent is 2:5.

8. A crystalline form CSI of Compound I, wherein the X-ray powder diffraction pattern comprises characteristic peaks at 2 theta values of 8.5°±0.2°, 12.7°±0.2° and 13.9°±0.2° using CuKα radiation.

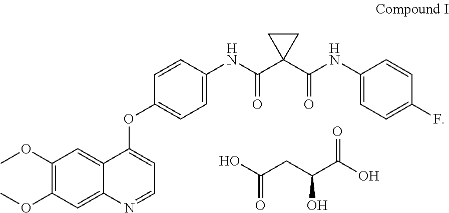

9. The crystalline form CSI of Compound I according to claim 8, wherein the X-ray powder diffraction pattern comprises one or two or three characteristic peaks at 2 theta values of 12.1°±0.2°, 17.9°±0.2° and 19.9°±0.2° using CuKα radiation.

10. The crystalline form CSI of Compound I according to claim 8, wherein the X-ray powder diffraction pattern comprises one or two or three characteristic peaks at 2 theta values of 14.9°±0.2°, 16.7°±0.2° and 25.5°±0.2° using CuKα radiation.

11. A process for preparing of crystalline form CSI of Compound I according to claim 8, wherein the process comprising:
Method 1: dissolving Compound I in acetic acid or a solvent mixture of acetic acid and an aromatic hydrocarbon, then rapidly evaporating at 50-80° C.; or
Method 2: dissolving the Compound I in acetic acid, a mixture of acetic acid and an aromatic, a mixture of acetic acid and an alkane, or a mixture of acetic acid and water; then adding an aromatic hydrocarbon, an alkane, an ester or a ketone into the solution with stiffing to obtain crystalline form CSI.

12. The process for preparing of crystalline form CSI of Compound I according to claim 11, wherein said aromatic hydrocarbon in Method 1 is toluene, the volume ratio of said acetic acid and toluene is 2:1-1:3.

13. The process for preparing of crystalline form CSI of Compound I according to claim 12, wherein the volume ratio of said acetic acid and toluene is 1:1.

14. The process for preparing of crystalline form CSI of Compound I according to claim 11, wherein the volume ratio of said acetic acid and aromatic, acetic acid and alkane, or acetic acid and water in Method 2 is 2:1-1:3.

15. The process for preparing of crystalline form CSI of Compound I according to claim 11, wherein in Method 2, said aromatic hydrocarbon is toluene, said alkane is n-heptane, said ester is isopropyl acetate, and said ketone is methyl isobutyl ketone.

16. The process for preparing of crystalline form CSI of Compound I according to claim 11, wherein said stiffing in Method 2 is at 0-5° C.

17. A crystalline form CSIII of Compound I of Compound I, wherein the X-ray powder diffraction pattern comprises characteristic peaks at 2 theta values of 8.5°±0.2°, 21.3°±0.2°, and 23.0°±0.2° using CuKα radiation.

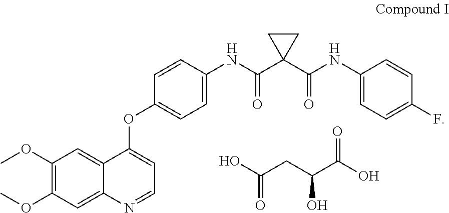

18. The crystalline form CSIII of Compound I according to claim 17, wherein the X-ray powder diffraction pattern comprises one or two or three characteristic peaks at 2 theta values of 14.4°±0.2°, 17.8°±0.2°, and 12.6°±0.2° using CuKα radiation.

19. The crystalline form CSIII of Compound I according to claim 17, wherein the X-ray powder diffraction pattern comprises one or two or three characteristic peaks at 2 theta values of 20.5°±0.2°, 24.0°±0.2°, and 16.4°±0.2° using CuKα radiation.

20. A process for preparing crystalline form CSIII of Compound I according to claim 17, wherein the process comprising:
Method 1: dissolving Compound I in an acid, a solvent mixture of an acid and an aromatic hydrocarbon, a solvent mixture of an acid and an alkane, or a solvent mixture of an acid and water, adding an aromatic hydrocarbon, an alkane, an ester or a ketone into the solution with stiffing to obtain a precipitate, then slurring the obtained solid in a solvent mixture of an aromatic hydrocarbon and water, separation again to obtain the crystalline form CSIII; or
Method 2:
step 1: dissolving the Compound I in an acid, stiffing and heating until the solid is completely dissolved, then naturally cooling the system to room temperature and filtering;
step 2: adding an aromatic hydrocarbon in the clear solution dropwise, then transferring the mixture to an environment at 0-10° C. and continuing stiffing, filtering the mixture to separate the solid, and drying;
step 3: heating the solid to 50-100° C. under nitrogen purging, and then cooling to 30° C. to obtain Form CSIII.

21. The process for preparing crystalline Form CSIII of Compound I according to claim 20, wherein the volume ratio of said acid and aromatic hydrocarbon, acid and alkane, or acid and water in Method 1 is 2:1-1:3.

22. The process for preparing crystalline form CSIII of Compound I according to claim 20, wherein in Method 1, said acid is acetic acid, said aromatic hydrocarbon is toluene, said alkane is n-heptane, said ester is isopropyl acetate, and said ketone is methyl isobutyl ketone.

23. The process for preparing crystalline form CSIII of Compound I according to claim 20, wherein in Method 2, said acid is acetic acid, said aromatic hydrocarbon is toluene.

24. The process for preparing crystalline form CSIII of Compound I according to claim 20, wherein said stiffing in step 1 of Method 2 is performed at 80° C.; said stiffing in step 2 is performed at 5° C., the time of said stiffing in step 2 is 10-20 hours, and said heating in step 3 is up to 100° C.

25. A pharmaceutical composition, wherein the pharmaceutical composition comprises a therapeutically effective amount of crystalline form CSI of Compound I according to claim 8, and a pharmaceutically acceptable carrier, a dilution agent or an excipient.

26. A method for treating a disease associated with inhibition of MET, VEGFR1/2/3, ROS1, RET, AXL, NTRK, or KIT, comprising administering to a subject in need thereof a therapeutically effective amount of crystalline form CSI of Compound I according to claim 8.

27. A method for treating thyroid cancer, lung cancer, gastric cancer, or liver cancer, comprising administering to a subject in need thereof a therapeutically effective amount of crystalline form CSI of Compound I according to claim 8.
